(19) United States
(12) Patent Application Publication
(10) Pub. No.: US 2013/0005588 A1
(43) Pub. Date: Jan. 3, 2013

(54) METHOD FOR DETERMINING SUBSTANCE NON-TOXICITY

(76) Inventor: Thomas Hartung, Baltimore, MD (US)

(21) Appl. No.: 13/635,008

(22) PCT Filed: Mar. 15, 2011
(86) PCT No.: PCT/US11/28525

§ 371 Date: Sep. 14, 2012

Related U.S. Application Data
(60) Provisional application No. 61/313,835, filed on Mar. 15, 2010.

(51) Int. Cl. G06F 19/00; G01N 33/566; G01N 30/00; C40B 20/00; G01R 33/46; C12Q 1/68; C40B 30/06

(52) U.S. Cl. 506/2; 435/6.12; 435/7.1; 506/10; 324/309; 73/61.52; 702/19

(57) ABSTRACT

Described herein are methods for establishing the non-toxicity of a substance. For example, described herein are methods for the construction of a comprehensive database of toxicity associated pathways and methods of using such a database.

### RELATED APPLICATIONS

This application claims the benefit of priority under 35 U.S.C. §119(e) to Provisional Application Ser. No. 61/313,835 filed Mar. 15, 2010, the contents of which are incorporated by reference in its entirety.

### BACKGROUND

The toxic effects of numerous environmental and consumer product chemicals have a large impact on human health. This important fact prompted the “National Conversation on Public Health and Chemical Exposures” by the CDC. Two independent studies suggest that we lack the necessary toxicity data for 86% of chemicals currently on the market. This is a serious public health issue. In 2007, the National Research Council (NRC) released the report “Toxicity Testing in the 21^{st} Century: A Vision and a Strategy”, that charted a long-range strategic plan for transforming toxicity testing. The report summarized the inadequacies of the current system, which relies on the use of a patchwork of 40+-year-old animal tests that are expensive (costing more than 3 billion dollars per year), time-consuming, have low-throughput and often provide results of limited predictive value. The low-throughput of current toxicity testing approaches (which are largely the same for industrial chemicals, pesticides and drugs) has led to a backlog of more than 80,000 chemicals whose potential toxicity remains largely unknown, and hinders toxicity testing in drug development.

The scientific understanding of how genes, proteins, and small molecules interact to form molecular pathways that maintain cell function is evolving rapidly. Pathways that lead to adverse health effects when perturbed are referred to as pathways of toxicity (PoT). The exploding scientific knowledge of mode of action in target cells, tissues and organs, driven by advances in molecular and computational tools, coupled with the concomitant development of high-throughput and high-content screening assays enable interrogation of these PoT and provide a means to study and evaluate the effects of thousands of chemicals. A number of PoT have already been identified; however, most PoT are only partially known and no common annotation exists.

There is a great need for novel methods useful in the mapping the entirety of these pathways, i.e. The Human Toxome, and new methods of using this pathway map to determine whether substances are non-toxic.

### SUMMARY

Disclosed herein are novel methods useful for establishing the non-toxicity of a substance or a mixture of substances. Generally, the methods described herein are based on a strategy of mapping the entirety of the finite number of metabolic pathways that contribute to toxicity when perturbed. Thus, disclosed herein are methods for generating a toxicity-associated pathway database and methods of using such a database to establish the non-toxicity of a substance.

In certain embodiments, the present invention relates to a method for generating a toxicity-associated pathway database from metabolic phenotype changes. In some embodiments this method includes the steps of: contacting a test cell population with a toxic substance; performing one or more assays to detect a modulation of metabolism in the contacted cell population, wherein the one or more assays detect, for example, the gene expression, gene regulation, protein expression, protein modification or metabolite production of the test cell population; identifying a toxic substance associated pathway based on the modulation of metabolism in the contacted cell population; and/or adding the toxic substance associated pathway to a database of toxicity-associated pathways. In some embodiments the steps of the process are repeated for a plurality of distinct toxic substances and a plurality of distinct cell populations. In certain embodiments, the invention relates to a database generated according to this method.

In some embodiments the present invention relates to a method for determining whether a test substance is non-toxic at a particular concentration. In certain embodiments this method includes the step of performing one or more assays that detect the modulation of a plurality of toxicity-associated metabolic pathways by the test substance to generate a toxicity-associated pathway phenotype for the test substance. In some embodiments the method also includes the steps of comparing the toxicity-associated pathway phenotype of the substance with a database of toxicity-associated pathways associated with toxic substances and determining whether the test substance is non-toxic at the concentration.

In some embodiments, the invention relates to a method for predicting the non-toxicity of a concentration of a test substance in an organism comprising the steps of performing one or more assays that detect the modulation of a plurality of toxicity-associated metabolic pathways and toxicity defense pathways by the concentration of the test substance to generate a toxicity-associated pathway and toxicity defense pathway phenotype for the concentration of the test substance; comparing the toxicity-associated pathway and toxicity defense pathway phenotype of the concentration of the test substance with a database of toxicity-associated pathways and toxicity defense pathways associated with toxic substances; and c) predicting the non-toxicity of the compound in the organism when toxicity-associated pathways are not perturbed or toxicity defense pathways are activated at the concentration of the test substance.

In some embodiments, the invention relates to a computer program product for determining whether a test substance or a mixture of test substances is non-toxic at a concentration, said computer program product residing on a non-transitory computer readable medium having a plurality of instructions stored thereon which, when executed by a computer processor, cause that computer processor to a) compare a toxicity-associated pathway phenotype of the concentration of the test substance with a database of toxicity-associated pathways associated with toxic substances, said toxicity-associated pathway phenotype for the concentration of the test substance having been generated by performing one or more assays that detect the modulation of a plurality of toxicity-associated metabolic pathways by the concentration of the test substance; and b) determine whether the test substance is non-toxic at the concentration.

In certain embodiments, at least a portion of the one or more assays are performed on a test cell population. In other embodiments, at least a portion of the one or more assays are performed on a plurality of test cell populations. In still other embodiments, the one or more assays includes a gene expression microarray assay, high-throughput sequencing, chromatography-mass spectrometry or an NMR assay. In yet another embodiment, at least a portion of the test cell population is lysed prior to performing one or more assays. In other embodiments, performing one or more assays comprises performing one or more assays that detect modulation of toxicity defense pathways. In other embodiments, the database comprises both toxicity associated pathways and toxicity defense pathways. In other embodiments, the test substance is determined to be non-toxic at the concentration if no toxicity-associated pathways were modulated by the test substance. In yet another embodiment, step a) and b) are performed on a range of concentrations of the test substance. In yet another embodiment, the invention the computer program product further comprises instructions for determining the concentration at which the test substance is no longer toxic.

In other embodiments, the invention relates to a computer program product for predicting the non-toxicity of a concentration of a test substance in an organism, said computer program product residing on a non-transitory computer readable medium having a plurality of instructions stored thereon which, when executed by a computer processor, cause that computer processor to: a)compare the toxicity-associated pathway and toxicity defense pathway phenotype of the concentration of the test substance with a database of toxicity-associated pathways and toxicity defense pathways associated with toxic substances, said toxicity-associated pathway and toxicity defense pathway phenotype for the concentration of the test substance having been generated by performing one or more assays that detect the modulation of a plurality of toxicity-associated metabolic pathways and toxicity defense pathways by the concentration of the test substance; and b) predict the non-toxicity of the compound in the organism when toxicity-associated pathways are not perturbed or toxicity defense pathways are activated at the concentration of the test substance.

### BRIEF DESCRIPTION OF THE FIGURES

FIG. 1 shows rat 3-D primary aggregating brain cell cultures under control conditions (non-treated) characterized by gene expression related to neuronal and glial proliferation, differentiation and maturation from 1 DIV to 35 DIV. The changes in gene expression levels quantified by real-time PCR of the neural precursor marker nestin (A), the neuronal marker NF-200 (B), the astrocytic marker S100β (C) and the oligodenrocytic marker MBP (D). Gene expression levels were normalized to the standard calibrator, the housekeeping gene 18S rRNA and the mRNA expression at 1 DIV. Data represent mean ±S.E.M. of three independent experiments performed in duplicates. **P<0.01 and ***P<0.001 comparing to 1 DIV.

FIG. 2 shows rat 3-D primary aggregating brain cell cultures under control conditions (non-treated) characterized by protein expression related to neural differentiation and maturation from 7 DIV to 21 DIV. The protein expression of the neural precursor marker nestin by western blot (A), quantified (C) remained stable over time. The protein expression of the neuronal marker NF-200 by western blot (B), quantified (D) significantly increased over time. Data represent mean±S.E.M. of one experiment performed in duplicates. *P<0.05 and ***P<0.001 comparing to 7 DIV.

FIG. 3 shows changes in gene expression induced by maneb exposure (0.1 μM, 1 μM and 10 μM) from 7 to 14 or 21 days in vitro (DIV). The housekeeping gene 18S (A) was stable over time. Note the down-regulation of the neural precursor marker nestin (B) already after exposure to 1 μM of maneb and the down-regulation of the neuronal marker NF-200 (C) already at the lower concentration of 0.1 μM maneb. There were no observed effects on the mRNA levels of the astrocytic marker S100β (D). Gene expression levels were normalized to the standard calibrator, the housekeeping gene 18S rRNA and the mRNA expression at 1 DIV. Data represent mean±S.E.M.*P<0.05 and ***P<0.001 comparing treated vs. control (non-treated).

FIG. 4 shows changes in gene expression induced by lead chloride exposure (0.1 μM, 1 μM and 10 μM) from 7 to 14 or 21 days in vitro (DIV). Note the up-regulation of the neural precursor marker nestin (A) after exposure to 10 μM of lead chloride and the down-regulation of the neuronal marker NF-200 (B) already at the lower concentration of 0.1 μM lead chloride. The mRNA levels of the astrocytic marker S100β (C) was up-regulated after exposure to 10 μM of lead chloride while the oligodendrocyte marker MBP (D) was down-regulated already after exposure to 0.1 μM of lead chloride. Gene expression levels were normalized to the standard calibrator, the housekeeping gene 18S rRNA and the mRNA expression at 1 DIV. Data represent mean±S.E.M.*P<0.05 and ***P<0.001 comparing treated vs. control (non-treated).

FIG. 5 shows a principle component analysis (PCA) plot of intra-cellular extracts of untreated controls and lead chloride treated aggregate samples 0.1 μM, 1 μM and 10 μM from 7 to 21 DIV.

FIG. 6 shows principle component analysis (PCA) plot of intra-cellular extracts of untreated controls and TCE treated aggregate samples 0.1 μM, 1 μM,10 μM and 50 μM from 7 to 21 DIV.

### DETAILED DESCRIPTION

### General

Described herein are methods for establishing the non-toxicity of a substance. In general, current toxicity assays are only able to establish whether a substance is toxic. Such assays are not capable of demonstrating the non-toxicity of a substance because, in such assays, the absence of a toxicity indication is insufficient to establish non-toxicity. However, the instant invention recognizes that non-toxicity of a substance could be established once the entirety of relevant pathways of toxicity were mapped. A method comprehensive of all relevant pathways of toxicity, showing that no relevant pathway is triggered, would ascertain the absence of toxicity of the substance or of a toxic substance at the given concentration. Described herein, for the first time are methods for the construction of a comprehensive database of toxicity associated pathways and methods of using such a database.

In certain embodiments, the invention relates to a novel combination of several established approaches (genetic and metabolic phenotyping, pattern recognition, systems biology) with novel techniques (database of toxicity pathways, testing strategy, data analysis procedure) for a new purpose (identification of non-toxicants). The methods described herein include the construction of a database of identified pathways, a test strategy (battery of combined tests) covering the relevant pathways and an algorithm to deduce whether the substance at the given concentration is non-toxic.

### Generating a Toxicity-Associated Pathway Database

In certain embodiments, the instant invention is related to a method for generating a toxicity-associated pathway database from metabolic phenotype changes comprising the steps of: a) contacting a test cell population with a toxic substance; b) performing one or more assays to detect a modulation of metabolism in the contacted cell population; c) identifying a toxic substance associated pathway based on the modulation of metabolism in the contacted cell population; d) adding the toxic substance associated pathway to a database of toxicity-associated pathways; and e) repeating steps a) through d) for a plurality of distinct toxic substances and a plurality of distinct cell populations. In some embodiments, the steps of the method are repeated until testing of new toxic substances no longer identify novel toxicity-associated pathways.

As used herein, the term “toxicity-associated pathway” refers to any cellular pathway that is modulated (i e inhibited, enhanced or altered) upon exposure of a cell to a toxic substance. Different cell populations (e.g., different cell types and/or cells from different organisms) may not have the exact same toxicity-associated pathways.

As used herein, the term “metabolic phenotype changes” refers to any changes that provide information regarding the metabolic state of the cell, including metabolite changes, protein changes, nucleic acid changes, carbohydrate changes, lipid changes, morphological changes, etc. Thus, any assay that provides information regarding the metabolism of the cell population can be performed in step b) of the above-described method, including, for example: assays that detect the presence, identity and/or level of metabolites; assays that evaluate gene expression and/or specific nucleic acid levels (including mRNA levels, miRNA levels, pre-miRNA levels, piRNA levels, rRNA levels etc.); assays that evaluate the epigenetic structure (e.g. DNA methylation, histone methylation, histone acetylation, histone ubiquitination, etc.) of particular genes and/or chromosomal locations (e.g., telomeres and/or centromeres); assays that detect the presence, identity and/or level of carbohydrates; assays that detect the presence, identity and/or level of lipids; and assays that evaluate protein processing, modification (e.g. phosphorylation, methylation, acetylation, ubiquitination etc.) and/or expression. In certain embodiments, the one or more assays performed in step b) may include a liquid chromatography-mass spectrometry (GC-MS) assay, a nuclear magnetic resonance (NMR) assay, a microarray assay (e.g. a nucleic acid or protein microarray), a nucleic acid sequencing assay (e.g., high-throughput sequencing assay, such as high throughput pyrosequencing), a flow-cytometry assay, a high-throughput microscopy assay, and/or a ChIP on chip assay.

The toxicity-associated pathway database can include any information related to the identified toxicity-associated pathways. Thus, in certain embodiments, the generated toxicity-associated pathway database includes the metabolite changes, nucleic acid (e.g., gene expression) changes, epigenetic changes, lipid changes, protein changes, carbohydrate changes, etc. associated with the toxicity-associated pathways.

In certain embodiments of the method at least a portion of the test cell population is lysed between step a) and step b). However, in certain embodiments a portion of the test cell population or the entire cell population is not lysed. In assays on intact cells, toxicity associated pathways can be identified based on, for example, molecules secreted by or expressed on the surface of the cells or through an otherwise outwardly detectable cellular phenotype, such as cell shape, size, motility or viability.

In some embodiments, for example, cells in culture can be exposed to a known toxic substance. Cells are lysed, for example, by replacing the cell culture medium with distilled water and/or methanol. After performance of a purification step to reduce non-metabolites in the lysate, LC-MS spectra are generated from samples obtained with different toxic and non-toxic substances and/or varied concentrations of toxic substances and are subjected to Principal Component Analysis. A signature of those signals contributing most to distinguish toxicants and non-toxicants or reflecting the concentration/response curve are deduced. As described above, analysis can also be carried out on the excreted metabolites into the cell culture medium without lysis. LC-MS can be substituted by and/or combined with other MS or NMR methodologies.

Cell systems useful in the above methods include, but are not limited to human primary cells/tissues, cell lines or cells/tissues derived from stem cells. Thus, the cell populations used may include whole or partial tissues, primary cells in culture and/or cell lines in culture. The cells may be obtained from any animal (including human) source that is amenable to primary culture and/or adaptation into cell lines. Lower organisms such as C. elegans can substitute as cell systems. In lieu of generating cell lines from animals, such cell lines may be obtained from, for example, American Type Culture Collection, (ATCC, Rockville, Md.), or any other Budapest treaty or other biological depository. The cells used in the assays may be from an animal (including human) source or may be recombinant cells tailored to express a particular characteristic. In certain embodiments the cells are derived from tissue obtained from humans or other primates, rats, mice, rabbits, sheep and the like. Techniques employed in mammalian primary cell culture and cell line cultures are well known to those of skill in that art. Indeed, in the case of commercially available cell lines, such cell lines are generally sold accompanied by specific directions of growth, media and conditions that are preferred for that given cell line.

In some embodiments, the methods disclosed herein also include a step of validating the identified toxic substance associated pathway using a second toxic substance having a known mode of action. Alternative and/or complementary measures of validation involve genetic information such as expression analysis of proteins linked to the metabolites identified, genetic variability leading to corresponding metabolic phenotypes or altered pattern response to toxicants, experimental interventions such as gene knock-out or gene-silencing and disease-associated genetic variation of metabolic phenotypes and response patterns. Primary measures include but are not limited to gene sequencing, mRNA expression, protein expression or phenotypic changes of cells as identified for example by image analysis.

The step of identifying a toxic substance associated pathway based on metabolism modulation can be accomplished by any technique known in the art. For example, by using bioinformatics, patterns associated with specific pathways of toxicity inducible by well-established toxins can be identified, while to the contrary untreated biological systems or systems treated with non-toxic reference compounds establish the physiological variability of metabolic phenotypes. Patterns and individual metabolite changes associated with one or more toxic substances can be used further on as biomarkers of this toxicity or of pharmacological effects, where such changes are desired. Thus, by using the existing knowledge of biochemical and physiological interactions of metabolites, pathways of toxicity can be deduced, i.e. by the consistent change of metabolites being associated in a known pathway. This analysis is further strengthened by similar results for similar toxic substances or similar alterations in pathways in conditions leading to similar phenotypes.

In certain embodiments, the methods described herein also include a step of correlating the identified toxic substance associated pathway with the test cell population's genetic profile. An individual's genetic make-up partially determines their reaction to substances including, but not limited to, their reaction to chemicals. In certain embodiments, the methods described herein are performed on a panel of similar cells, which differ in individual genes or groups of genes. By identifying cell responses specific to particular cell populations and tracing the responses to the specific genetic make-up of the effected cell, pathways of interaction of the substance within the cell system can be identified or the suspected pathways verified.

In some embodiments, a panel of genetically variant cells can be obtained by, for example: the combination or comparison of cells from different donor humans or animals; the combination or comparison of cells from donors with or without a certain disease; the induction of mutations in cells from one or more donors; the random or targeted insertion of genetic material and disruptors of genetic materials in the genome of cells from one or more donors; the recombination of genetic material of different donors; and/or the construction of artificial cells. A panel of cells can be brought into contact with test substances in parallel, after mixing or in sequence. Cellular responses including, but not limited to, cell death can be assessed. Abnormal responses, such as increased or decreased responses compared to the majority of cells or historic controls are used to identify those with a genetic makeup relevant for the identification of pathways of interaction. This includes, but is not limited to, the survival of a cell in the presence of an otherwise lethal concentration of the substance. In case of dividing cells, this might include, but is not limited to, the favored growth of a particular cell type in the presence of the substances. Other cell responses allowing the identification or isolation of cells with a genetic makeup that alters their response to a test substance can include, but is not limited to, cell image analysis and cell sorting.

In certain embodiments, the genetic variation linked to the variation in response is identified. This can be done by sequencing or by otherwise obtaining information on the genetic makeup of the cell of interest. If the cells differ in multiple aspects of their genetic makeup, consensus patterns of various cell variants can be used.

For example, in certain embodiments cells from different donors, such as blood leukocytes, are labeled with detectable markers (e.g. fluorescently labeled antibodies) before mixing them. Current flow cytometry cell analyzers can measure up to 16 colors in an individual cell, thus up to 2¹⁶ (65,536) distinct cell populations from individual donors could be detectably and distinctly labeled. Aberrant reactions to particular substances can be detected by the survival of particular cell types in the presence of an otherwise toxic substance or the absence of a toxicity induced response, such as apoptosis or early stress responses. The distinct labeling allows the response to be traced back to the cell donor's genetic makeup, such as mutations, polymorphisms (including single nucleotide polymorphisms, or SNPs), gene variations, whole genome sequences or disease states.

In another exemplary embodiment, a cell line can be randomly mutated using standard agents such as but not limited to ENU (N-ethyl-N-nitrosourea) or MNNG (N-methyl-N′-nitro-N-nitrosoguanidine). The mutated population of cells is exposed to toxic concentrations of a substance. Surviving cells are clonally expanded by creating colonies from individual cells. The genetic makeup of these clones is assessed, e.g. by sequencing. The information is used to deduce pathways impaired, especially from several variants showing the same resistance and originating from biochemically or physiologically connected genes.

Thus, in certain embodiments the invention allows the identification of genes impacting on the response of cells to substances on the basis of knowledge of pathways connecting these genes, their proteins and/or their metabolites and binding partners. This can be relevant for the identification of pathways of toxicity or pathways to target, manipulate or alter cell responses, such as drug-able pathways. This allows, for example, the identification of new substances by designing test systems representative of the pathway identified and the identification of modes of action (pathways) of toxic substances.

In some embodiments, the instant invention relates to a method for generating a toxicity defense pathway database. Such methods utilize the same assays as and basic techniques as were employed in the above described method for the generation of a toxicity-associated pathway database, but the assays are performed using non-toxic substances in a given cell population, rather than toxic substances.

Thus, in some embodiments the instant invention is related to a method for generating a toxicity defense pathway database from metabolic phenotype changes comprising the steps of: a) contacting a test cell population with a non-toxic substance; b) performing one or more assays to detect a modulation of metabolism in the contacted cell population; c) identifying a toxicity defense pathway based on the modulation of metabolism in the contacted cell population; d) adding the toxicity defense pathway to a database of toxicity defense pathways; and e) repeating steps a) through d) for a plurality of distinct non-toxic substances and a plurality of distinct cell populations. In some embodiments, the steps of the method are repeated until testing of new non-toxic substances no longer identify novel toxicity defense pathways. In some embodiments, the created database includes both toxicity-associated pathways and toxicity defense pathways.

In certain embodiments, the invention relates to the database created according to any of the above methods.

### Determining Substance Non-Toxicity

In certain embodiments, the instant invention relates to a method for determining whether a test substance is non-toxic at a concentration that includes the steps of: a) performing one or more assays that detect the modulation of a plurality of toxicity-associated metabolic pathways by the concentration of the test substance to generate a toxicity-associated pathway phenotype for the concentration of the test substance; b) comparing the toxicity-associated pathway phenotype of the concentration of the test substance with a database of toxicity-associated pathways (e.g., a database generated according to the methods described above); and c) determining whether the test substance is non-toxic at the concentration. In certain embodiments, substances that do not generate a metabolic phenotype indicative of toxicity will be considered non-toxic.

In some embodiments the method described above is performed using a plurality of concentrations of the test substance and concentrations of the test substance that do not result in metabolic phenotypes indicative of toxicity will be considered non-toxic concentrations. In such embodiments the method may also include the step of determining the concentration at which the test substance is no longer toxic.

As used herein, the term “test substance” refers to any potentially toxic substance or mixture of substances being evaluated according to the methods disclosed herein. Thus, the term “test substance” is interpreted broadly to encompass, for example, any molecule, including any biomolecule (e.g., any protein, nucleic acid, lipid, etc.), compound, mixture, complex, polymer, copolymer, chemical entity, composition, environmental contaminant, drug, metabolite, therapeutic agent, biological agent, etc.

In some embodiments, any assay that provides information regarding the metabolism of the cell population can be performed in step a) of the above-described method, including, for example: assays that detect the presence, identity and/or level of metabolites; assays that evaluate gene expression and/or specific nucleic acid levels (including mRNA levels, miRNA levels, pre-miRNA levels, piRNA levels, rRNA levels etc.); assays that evaluate the epigenetic structure (e.g. DNA methylation, histone methylations, histone acetylation, histone ubiquitination, etc.) of particular genes and/or chromosomal locations (e.g., telomeres and/or centromeres); assays that detect the presence, identity and/or level of carbohydrates; assays that detect the presence, identity and/or level of lipids; and assays that evaluate protein processing, modification (e.g. phosphorylation, methylation, acetylation, ubiquitination etc.) and/or expression. In certain embodiments, the one or more assays performed in step b) may include a liquid chromatography-mass spectrometry (GC-MS) assay, a nuclear magnetic resonance (NMR) assay, a microarray assay (e.g. a nucleic acid or protein microarray), a nucleic acid sequencing assay (e.g., high-throughput sequencing assay, such as high throughput pyrosequencing), a flow-cytometry assay, a high-throughput microscopy assay, and/or a ChIP on chip assay.

In some embodiments of the method described herein, at least a portion of the one or more assays performed in step a) are performed on a test cell population and/or a plurality of test cell populations. Cell systems useful in the above methods include, but are not limited to human primary cells/tissues, cell lines or cells/tissues derived from stem cells. Thus, the cell populations used may include whole or partial tissues, primary cells in culture and/or cell lines in culture. The cells may be obtained from any mammalian source that is amenable to primary culture and/or adaptation into cell lines. In lieu of generating cell lines from animals, such cell lines may be obtained from, for example, American Type Culture Collection, (ATCC, Rockville, Md.), or any other Budapest treaty or other biological depository. The cells used in the assays may be from an animal source or may be recombinant cells tailored to express a particular characteristic. In certain embodiments the cells are derived from tissue obtained from humans or other primates, rats, mice, rabbits, sheep and the like. Techniques employed in mammalian primary cell culture and cell line cultures are well known to those of skill in that art. Indeed, in the case of commercially available cell lines, such cell lines are generally sold accompanied by specific directions of growth, media and conditions that are preferred for that given cell line. In certain embodiments, cell culture protocols validated for the purpose of toxicity testing will be employed. This allows, for example, the use of the cell's respective substances and data interpretation procedures as adversity thresholds.

In certain embodiments of the method at least a portion of a test cell population is lysed before step a). However, in certain embodiments a portion of the test cell population or the entire cell population is not lysed. In assays on intact cells, toxicity associated pathways can be identified based on, for example, molecules secreted by or expressed on the surface of the cells or through an otherwise outwardly detectable cellular phenotype, such as cell shape, size, motility or viability.

In some embodiments, the method described herein also includes performing one or more assays that detect modulation of toxicity defense pathways (as described above). In general, in such embodiments the database will contain both toxicity associated pathways and toxicity defense pathways.

### EXEMPLIFICATION

The following example is included merely for purposes of illustration of certain aspects and embodiments of the present invention, and are not intended to limit the invention in any way.

### Example 1

### Pilot Study Modeling Developmental Neurotoxicity-Associated Pathways

### Rat Primary 3-D Aggregating Brain Cell Cultures

Generally one batch of Rat primary aggregating brain cell cultures aggregates were prepared every month. Cultures were maintained up to 35 days in vitro and did not display any loss in cell viability. Data obtained in the cultures showed a good reproducibility within a single batch and between independent batch preparations. The endpoints applied to study processes of neurodevelopment include quantitative real-time PCR, Western blot analysis and mass spectrometry based metabolomics.

### The Expression of Genes Relevant Neurodevelopment in Aggregating Brain Cell Cultures

Aggregate cultures were maintained for 35 days in vitro (DIV). Samples were taken at DIV 1, 7, 14, 21, 28, 35 and mRNA was isolated, purified and quantified by RT-PCR.

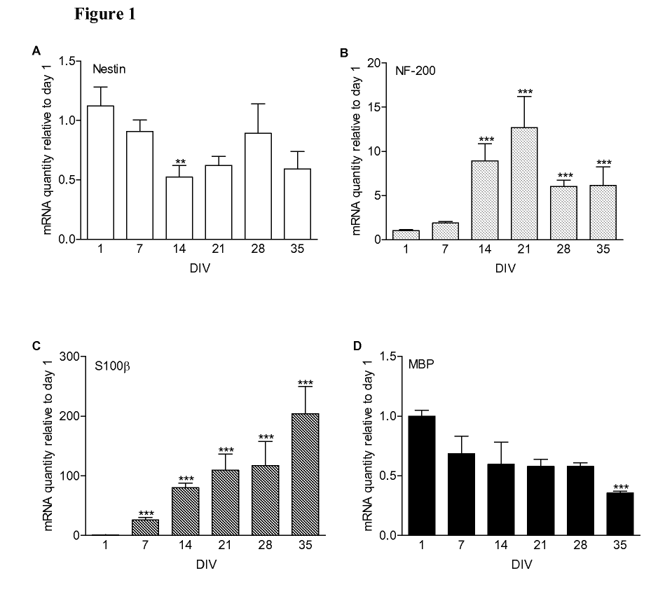

The expression of the following genes was quantified; nestin expressed in neural precurser cells, neurofilament-200 (NF-200) expressed in neurons, S100β expressed in astrocytes and myelin basic protein (MBP) expressed in oligodendrocytes. The expression of the respective genes during the different DIV is displayed in FIG. 1. Nestin expression (FIG. 1A) significantly decreases over time, which indicates a reduction in neural precurser cells during development. NF-200 (FIG. 1B) and S100β (FIG. 1C) expression significantly increases over time, which is likely due to the differentiation and maturation of neurons and proliferation and differentiation of astrocytes. The expression of myelin basic protein (FIG. 1D) remained stable until day 28, but decreased slightly at day 35.

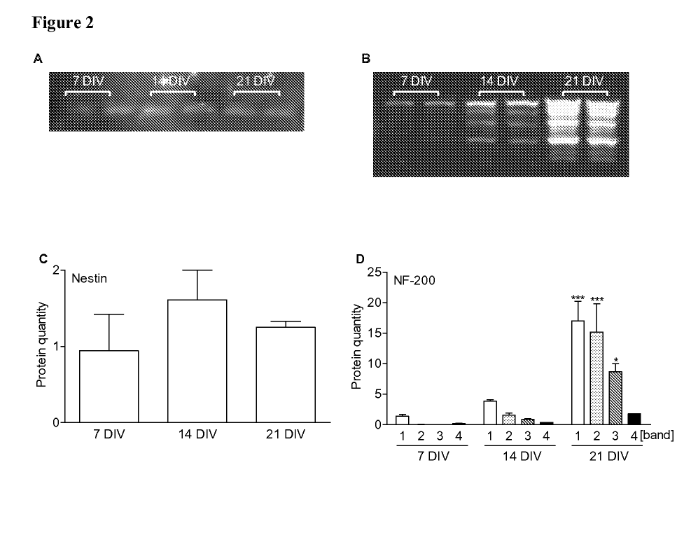

To confirm that the obtained gene expression results were translated to differences in protein levels, nestin and NF-200 proteins were quantified by Western blot analysis. The protein levels are displayed in FIG. 2. Nestin remained stable during developent and NF-200 increased over time, as seen for the gene expression. Thus, the obtained results demonstate processes of neurodevelopment in aggregating brain cell cultures. Results are consistent with previous data based on the quantitative measurement of cell type specific enzymes during the same period of neurodevelopment (data not shown).

### The Adverse Effects of Potential DNT Chemicals on Gene Expression

Aggregating brain cell cultures were exposed to potential DNT chemicals during both early (day in vitro 7-14) and late developmental stages (day in vitro 7-21). The chemicals with potential to induce developmental neurotoxicity (DNT) tested in the pilot assay are listed in Table 1. Chemicals were dissolved in water or DMSO depending on their chemical properties. Control cells received the vehicle without the DNT chemical. The final concentration of DMSO was 0.1%, which showed no significant effect on cell viability or gene expression. A summary of the results for the chemicals tested so far are provided in Table 2.

TABLE 1
DNT chemicals tested in aggregating brain cell cultures.

| Chemicals | Exposure | Toxic effects and/or main mechanisms of toxicity |
|---|---|---|
| Aspartame | Food additive | Excitotoxicity mainly through activation of the NMDA-R, |
| | | reduction of acethyl choline esterase (AchE) activity and |
| | | increase in reactive oxygen species (ROS). |
| Bisphenol A | Plastic additive | Endocrine disrupter at very low doses, can suppress cell |
| | | proliferation, can induce apoptotic cell death and produce |
| | | ROS. |
| Cadmium Chloride | Environmental | Causesoxidative stress and affects genes involved in cell |
| | contaminant, smoking | cycle regulation. |
| Carbaryl | Pesticide | Affects neurite outgrowth, inhibits nitric oxide synthesis |
| | | (NOS) and inhibits AchE. |
| Chlorpyrifos | Pesticide | Inhibits AchE, induces damage to RNA and DNA |
| | | synthesis, oxidative stress, astroglial proliferation and cell |
| | | differentiation. |
| Lamotrigine | Anti convulsant drug | Interferes with the voltage gated sodium channels and has |
| | | shown teratogenic effects in some studies. |
| Lead Chloride | Environmental | Associated with numerous adverse effects in the central |
| | contaminant | nervous system (CNS), including destruction of the blood |
| | | brain barrier, loss of neurons, gliosis and oxidative stress. |
| Lindane | Pesticide | Inhibits AchE, noradrenalin uptake, GABA |
| | | neurotransmission and blocksglycinereceptors. |
| Maneb | Pesticide | Inhibits GABA synthesis, causes loss of dopaminergic and |
| | | GABAergic neurons, decreases ATP levels and causes |
| | | oxidative stress. |
| Trichloroethylene | Environmental | Associated with adverse effects in the CNS, induces loss |
| | contaminant | of dopaminergic neurons and oxidative stress. |
| Valproic acid | Antiepileptic drug | Recognized as a teratogenic compound, modifies the |
| | | release of GABA. |

TABLE 2
The lowest concentration inducing changes in
mRNA levels of nestin, NF-200, S100β and MBP.

| Chemicals | Nestin | | NF-200 | | S100β | | MBP | |
|---|---|---|---|---|---|---|---|---|
| Aspartame | —¹ | | —¹ | | —¹ | | —¹ | |
| Bisphenol A | —¹ | | 100 | μM | —¹ | | —¹ | |
| Cadmium Chloride | 1 | μM | 1 | μM | 1 | μM | 10 | μM |
| Carbaryl | 0.1 | μM | 0.1 | μM | —¹ | | 0.1 | μM |
| Chlorpyrifos | 0.1 | μM | 0.1 | μM | 10 | μM | 1 | μM |
| Lamotrigine | —¹ | | —¹ | | —¹ | | No data² | |
| Lead Chloride | 10 | μM | 0.1 | μM | 10 | μM | 0.1 | μM |

| | | | | | | | |
|---|---|---|---|---|---|---|---|
| Lindane | 0.1 | μM | 0.1 | μM | —¹ | 1 | μM |
| Maneb | 1 | μM | 0.1 | μM | —¹ | 0.1 | μM |

| | | | | | | |
|---|---|---|---|---|---|---|
| Trichloroethylene | 50 | μM | 0.1 | μM | —¹ | No data² |

| | | | | | | |
|---|---|---|---|---|---|---|
| Valproic acid | —¹ | —¹ | 500 | μM | 100 | μM |

¹no significant changes
²gene not tested

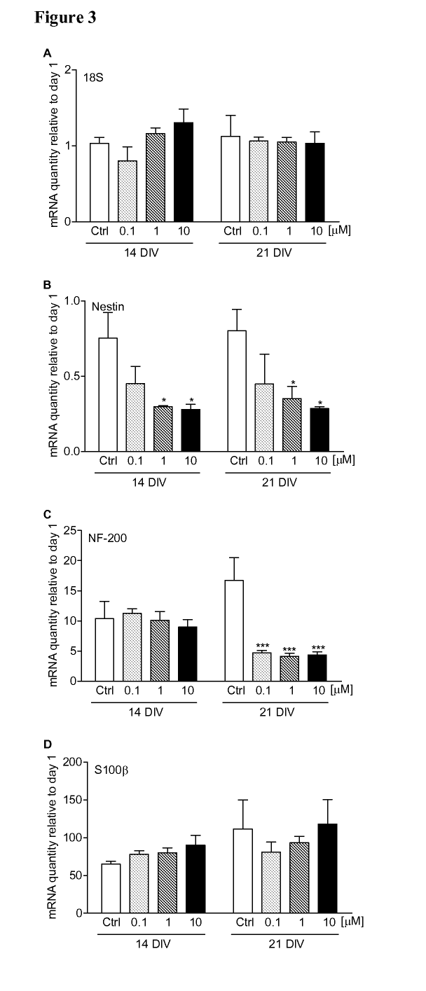

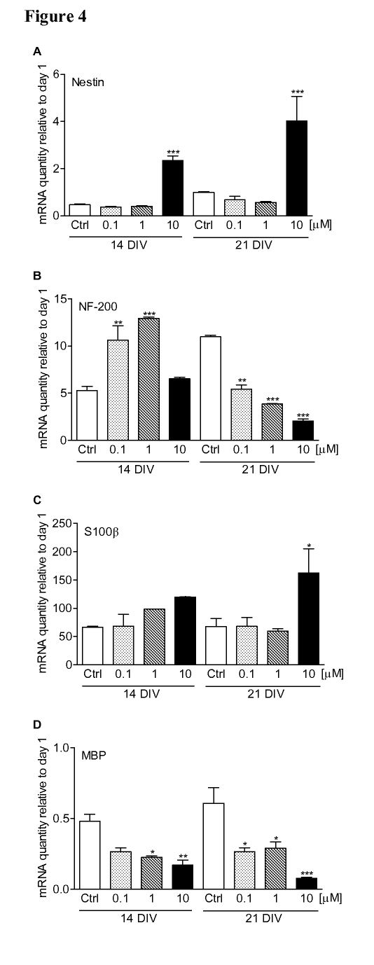

The potential DNT chemicals that did not induce any significant effects on quantified gene expression levels include aspartame and lamotrigine. Chemicals that significantly affected the expression of one or several cell type related genes include: bisphenol A, cadmium chloride, carbaryl, chloropyrifos, lead chloride, lindane, maneb, trichloroethylene and valproic acid. A summary of the results is given in Table 2, and the graphs of maneb and lead chloride are provided in FIGS. 3 and 4.

### Metabolomics Method for the Analysis of Aggregating Brain Cell Cultures

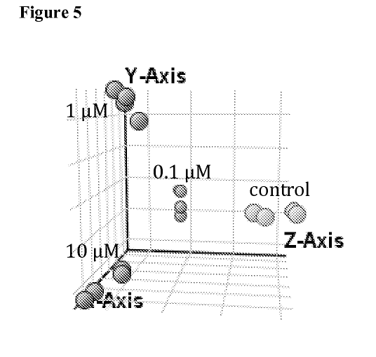

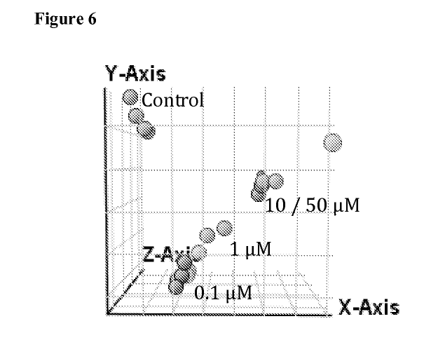

Cellular extracts (control and treated cultures) were prepared as described. The samples were used to establish an LC-MS method for the analysis of metabolites. Results showed that control and treated samples (lead chloride and TCE) could be distinguished from each other and induced concentration response effects (FIGS. 5 and 6). Furthermore, the data showed that the treatment with the two chemicals altered hundreds of metabolites in the aggregate samples. Significant pertubations in metabolite levels were determined by principal component analysis and metabolites were identified by a Metlin metabolite database search using their retention time and accurate mass. Examples of metabolites that were perturbed by lead chloride and TCE treatment in the neuronal cell model and could be linked to biological pathways are provided in Table 3. Thus, the first analysis of aggregate samples demonstrated the proof of principle to detect metabolic alterations due to toxic treatment in aggregating brain cell cultures allowing to link to toxicity associated pathways.

TABLE 3
Examples of perturbed metabolites in primary rat
aggregating brain cell cultures by treatments with
lead chloride and trichloroethylene for 14 days.

| Perturbed metabolites | Associated biological pathways |
|---|---|
| Glutathione | Oxidative stress |
| ATP | Citric acid cycle |
| ADP | Citric acid cycle |
| Creatine | Energy metabolism/apoptosis/cell |
| | differentiation |
| Colbalamin (Vitamin B12) | Methionine/Folic acid synthesis |
| Pyridoxal (Vitamin B6) | Neurotransmitter synthesis |
| UDP-N-acetyl-D-galactosamine | Glucose metabolism |
| Fructose-1-phosphate | Glucose metabolism |
| Nicotinic acid adenine | Ca(2+) signaling |
| dinucleotide | |
| Heparan sulfate | Fibroblast growth factor 2 signaling/ |
| | Extracellular matrix remodeling |
| Resolvin D1 | Biosynthetic pathway to neuroprotectin/ |
| | Inflammation |

### EQUIVALENTS

While specific embodiments of the subject invention have been discussed, the above specification is illustrative and not restrictive. Many variations of the invention will become apparent to those skilled in the art upon review of this specification. The appended claims are not intended to claim all such embodiments and variations, and the full scope of the invention should be determined by reference to the claims, along with their full scope of equivalents, and the specification, along with such variations.

All publications and patents mentioned herein are hereby incorporated by reference in their entirety as if each individual publication or patent was specifically and individually indicated to be incorporated by reference. In case of conflict, the present application, including any definitions herein, will control.

1-25. (canceled)

26. A computer program product for predicting the non-toxicity of a concentration of a test substance in an organism, said computer program product residing on a non-transitory, computer-readable medium having a plurality of instructions stored thereon that, when executed by a computer processor, cause the computer processor to carry out the steps comprising:
a) receive an indication of a cellular reaction induced by a test substance and a given concentration of the test substance;
b) access a database of substances mapped to relevant toxicity-associated pathways, wherein the toxicity-associated pathways correspond to cellular pathways that are modulated upon exposure of a cell to a given substance;
c) compare the test substance to the database to determine that no relevant toxicity-associated pathway would be triggered by the test substance at the given concentration of the test substance; and
d) indicate at least one of an absence of toxicity of the substance and an absence of the toxicity of the substance at the test concentration.

27. The computer program product of claim 26 wherein the database includes data regarding at least one of metabolite changes, nucleic acid changes, epigenetic changes, lipid changes, protein changes, and carbohydrate changes, associated with the toxicity-associated pathways.

28. The computer program product of claim 26 wherein the database includes data regarding variations in cell populations, including at least one of cell types and organism types, mapped to the relevant toxicity-associated pathways.

29. The computer program product of claim 26 further causing the computer processor to identify toxicity pathway changes impacting on the response of cells to the test substance based on at least one of pathways connecting genes, proteins associated with the genes, and metabolites and binding partners associated with the toxicity-associated pathways.

30. The computer program product of claim 26 wherein step c) further includes comparing the test substance to the database to determine a toxicity defense pathway phenotype for the test substance at the given concentration of the test substance

31. The computer program product of claim 30 wherein step d) further includes Indicating a toxicity defense condition induced by the substance at the test concentration.

32. The computer program product of claim 30 further comprising e) generate a toxicity-associated pathway phenotype for the concentration of the test substance and store the toxicity-associated pathway phenotype in the database.

33. The computer program product of claim 26 wherein the toxicity-associated pathways are correlated from metabolic phenotype changes.

34. The computer program product of claim 33 wherein metabolic phenotype changes include at least one of metabolite changes, protein changes, nucleic acid changes, carbohydrate changes, lipid changes, and morphological changes.

35. A method for determining whether a test substance or a mixture of test substances is non-toxic at a given concentration, the comprising the steps of:
a) performing at least one assay to detect the modulation of a plurality of toxicity-associated metabolic pathways using the given concentration of the test substance to generate a toxicity-associated pathway phenotype for the given concentration of the test substance;
b) comparing the toxicity-associated pathway phenotype of the given concentration of the test substance with a database of toxicity-associated pathways associated with toxic substances; and
c) from the comparison of step b), determining whether the test substance is non-toxic at the given concentration.

36. The method of claim 35 wherein the at least one assay performed in step a) is performed on a test cell population.

37. The method of claim 36 wherein at least one assay performed in step a) includes at least one of a gene expression microarray assay, a high-throughput sequencing, a chromatography-mass spectrometry, and an NMR assay.

38. The method of claim 37 wherein at least a portion of the test cell population is lysed prior to step a).

39. The method of claim 35 wherein step a) further includes performing at least one assay that detects the modulation of a plurality of toxicity defense pathways by the given concentration of the test substance to generate a toxicity defense pathway phenotype for the concentration of the test substance.

40. The method of claim 39 wherein step b) includes comparing the toxicity defense pathway phenotype of the given concentration of the test substance with a database of toxicity defense pathways associated with toxic substances and step c) includes predicting the non-toxicity of the test substance when toxicity defense pathways are activated at the given concentration of the test substance.

41. A method for predicting the non-toxicity of a given concentration of a test substance in an organism, the method comprising the steps of:
a) performing at least one assay to detect the modulation of at least one toxicity defense pathway by the given concentration of the test substance to generate a toxicity defense pathway phenotype for the given concentration of the test substance;
b) comparing the toxicity defense pathway phenotype of the given concentration of the test substance with a database of toxicity defense pathways associated with toxic substances; and
c) predicting the non-toxicity of the test substance in the organism when toxicity defense pathways are not activated at the given concentration of the test substance.

42. The method of claim 41 wherein step a) includes performing at least one assay to detect the modulation of a plurality of toxicity-associated metabolic pathways using the given concentration of the test substance to generate a toxicity-associated pathway phenotype for the given concentration of the test substance.

43. The method of claim 42 wherein step b) includes comparing the toxicity-associated pathway phenotype of the given concentration of the test substance with a database of toxicity-associated pathways associated with toxic substances.

44. The method of claim 43 wherein step c) includes determining, from the comparison of step b), whether the test substance is non-toxic at the given concentration.

45. The method of claim 41 wherein the database includes data regarding at least one of metabolite changes, nucleic acid changes, epigenetic changes, lipid changes, protein changes, and carbohydrate changes, associated with the toxicity defense pathway.
